(19) United States
(12) Patent Application Publication
(10) Pub. No.: US 2020/0376431 A1
(43) Pub. Date: Dec. 3, 2020

(54) METHODS AND APPARATUS TO DEODORIZE FLUIDS

(71) Applicant: Durr UNIVERSAL, Inc., Stoughton, WI (US)

(72) Inventors: Matthew Holschuh, Fitchburg, WI (US); Adam Shambeau, Stoughton, WI (US)

(21) Appl. No.: 16/862,099

(22) Filed: Apr. 29, 2020

Related U.S. Application Data
(60) Provisional application No. 62/854,679, filed on May 30, 2019.
(60) Provisional application No. 62/854,680, filed on May 30, 2019.

(51) Int. Cl. B01D 53/78; A61L 9/14

(52) U.S. Cl. CPC B01D 53/78; A61L 2209/134; A61L 2209/111; A61L 9/145

(57) ABSTRACT

Methods and apparatus to deodorize fluids are disclosed. A disclosed example apparatus for deodorizing fluid from a fluid distributor includes a valve to enable the fluid from to enter a relief line from the fluid distributor, the relief line including an outlet, a bleed line to receive the fluid, the bleed line fluidly coupled to the relief line, a pressure tank fluidly coupled to the bleed line, the pressure tank to store a deodorant, and a deodorant line fluidly coupled between the pressure tank and the outlet, the fluid in the bleed line to urge the deodorant to the deodorant line.

### RELATED APPLICATIONS

This patent claims priority to, and the benefit of, U.S. Provisional Patent Application Ser. No. 62/854,679 and U.S. Provisional Patent Application Ser. No. 62/854,680, both of which were filed on May 30, 2019. U.S. Provisional Patent Application Ser. No. 62/854,679 and U.S. Provisional Patent Application Ser. No. 62/854,680 are hereby incorporated herein by reference in their entireties.

### FIELD OF THE DISCLOSURE

This disclosure relates generally to industrial processing of fluids and, more particularly, to methods and apparatus to deodorize fluids.

### BACKGROUND

Natural gas distribution systems typically involve compressed natural gas that flows through a pipeline that can extend through multiple miles. Blowdown events occur when the compressed natural gas is vented or released into the environment, thereby causing a pressure of the natural gas to equalize with atmospheric pressure. These blowdown events can be initiated for maintenance or emergencies.

For security reasons, natural gas, as well as other kinds of combustible gases including propane, butane or other carbon based gases or mixtures thereof distributed through pipelines or stored in storing facilities (e.g. storage tanks, gas containers, etc.) are admixed with an odorant. This odorant can cause strong odors when released into the atmosphere during blowdown events, for example.

### SUMMARY

An example apparatus includes a valve to enable the fluid to enter a relief line from the fluid distributor, the relief line including an outlet, a bleed line to receive the fluid, the bleed line fluidly coupled to the relief line, a pressure tank fluidly coupled to the bleed line, the pressure tank to store a deodorant, and a deodorant line fluidly coupled between the pressure tank and the outlet, the fluid in the bleed line to urge the deodorant to the deodorant line.

An example method includes opening a valve of a relief line that is fluidly coupled to a fluid distributor, directing fluid from the relief line to an outlet, and directing fluid from the relief line to a bleed line and into a pressure tank that stores a deodorant, the fluid from the bleed line to urge the deodorant to flow toward the fluid of the relief line.

An example blowdown system to be used with a fluid distributor includes a relief line, a valve to enable fluid to flow from the fluid distributor and into a relief line, a reaction chamber fluidly coupled to the relief line, and a pressure tank fluidly coupled to the bleed line, the pressure tank to store a deodorant to be supplied to a nozzle of the reaction chamber, the deodorant to be urged toward the nozzle by the fluid in the bleed line.

### BRIEF DESCRIPTION OF THE DRAWINGS

FIG. 1 illustrates an example blowdown system in accordance with teachings of this disclosure.

FIG. 2 is a detailed view of the example blowdown system of FIG. 1.

FIG. 3 illustrates an alternative example blowdown system.

FIG. 4 illustrates another alternative example blowdown system.

FIG. 5 is a schematic overview of an example blowdown control system that can be implemented in examples disclosed herein.

FIG. 6 is a flowchart representative of an example method to implement examples disclosed herein.

FIG. 7 is a block diagram of an example processing platform structured to execute the example method of FIG. 6 to implement examples disclosed herein.

The figures are not to scale. Instead, the thickness of the layers or regions may be enlarged in the drawings. In general, the same reference numbers will be used throughout the drawing(s) and accompanying written description to refer to the same or like parts. As used in this patent, stating that any part is in any way on (e.g., positioned on, located on, disposed on, or formed on, etc.) another part, indicates that the referenced part is either in contact with the other part, or that the referenced part is above the other part with one or more intermediate part(s) located therebetween. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Stating that any part is in “contact” with another part means that there is no intermediate part between the two parts.

Descriptors “first,” “second,” “third,” etc. are used herein when identifying multiple elements or components which may be referred to separately. Unless otherwise specified or understood based on their context of use, such descriptors are not intended to impute any meaning of priority, physical order or arrangement in a list, or ordering in time but are merely used as labels for referring to multiple elements or components separately for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for ease of referencing multiple elements or components.

### DETAILED DESCRIPTION

Methods and apparatus to deodorize fluids are disclosed. Blowdown events occur when compressed natural gas is vented or released into the environment from a pipeline, a storage tank, a fluid delivery system and/or a fluid containment system. The natural gas is released into the environment until a pressure of the natural gas equalizes with atmospheric pressure. However, the release of the natural gas into the environment can result in unwanted odors.

Examples disclosed herein enable effective removal (i.e., deodorizing) of odors that can be released from blowdown events. In particular, examples disclosed herein utilize a relief line that is fluidly coupled to a fluid distributor, such as a pipeline for example. The relief line includes and/or is fluidly coupled to a valve (e.g., a shut off valve, a bypass valve, a gate valve, etc.) that supplies the fluid to an outlet and a bleed line. The bleed line, in turn, is fluidly coupled to a pressure tank, which stores a deodorant. Further, the pressure tank is fluidly coupled to a deodorant line that supplies the deodorant to the outlet. In particular, the fluid of the bleed line generates a pressure in the pressure tank and, thus, urges the deodorant toward the deodorant line and, thus, the outlet. Accordingly, the fluid exiting the outlet is deodorized. The fluid can be a mixture of a main fluid (e.g. natural gas, compressed natural gas, liquid natural gas, other combustible gases, such as butane, propane, etc.] and/or mixtures thereof) and at least one odorant admixed to the main fluid (e.g., for security reasons).

In some examples, the outlet includes and/or is fluidly coupled to a reaction chamber. In some such examples, the reaction chamber functions as a sound silencer. In some examples, the reaction chamber is positioned and/or disposed within the sound silencer. Additionally or alternatively, the deodorant is dispersed to the outlet and/or the reaction chamber via a spray nozzle (e.g., a mist nozzle, a sprayer, a mist sprayer, etc.). In some examples, a holding tank (e.g., a bulk holding tank) is implemented to supply the fluid to the pressure tank. In some examples, the deodorant is a liquid deodorant that removes or masks odors and/or marcaptans in the fluid.

As used herein, the term “fluid distributor” refers to a pipeline, fluid delivery device, a storage tank, and/or a holding tank, a reservoir, ductwork, and/or a pipeline, etc. As used herein, stating that an object is “above” or “elevated” means that the object is vertically above in height in relation to gravity. As used herein, the terms “blowdown event” and “blowdown” refer to an action, step and/or process related to lowering a pressure of a fluid in a fluid distributor. Accordingly, the terms “blowdown event” and “blowdown” can refer to an operation that is manually initiated, based on a control system or based on a fluid condition within the fluid distributor (e.g., a pressure threshold being exceeded in the fluid distributor).

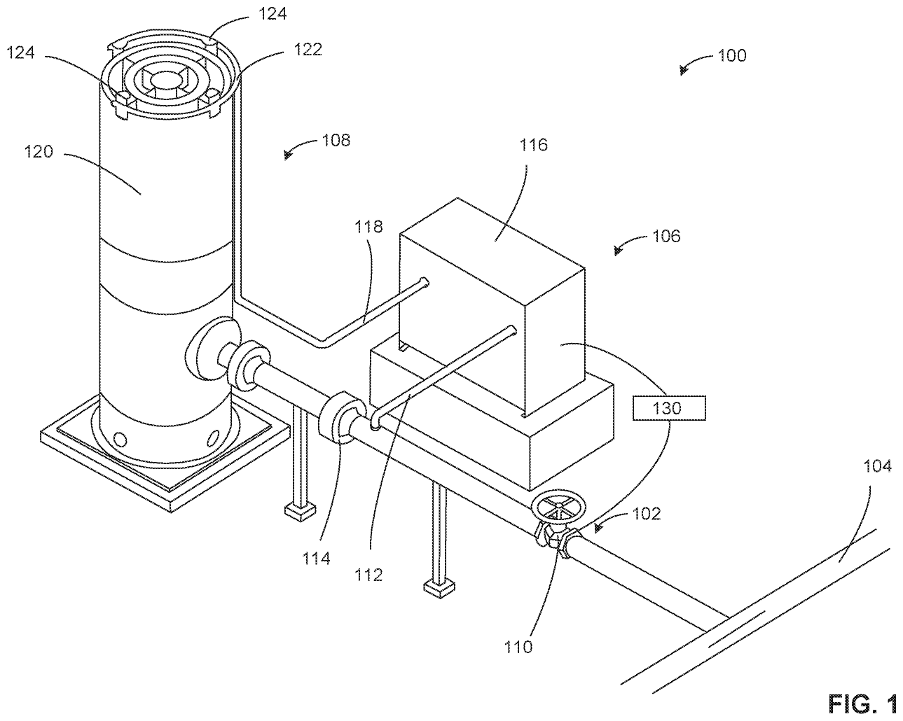

FIG. 1 illustrates an example blowdown system 100 in accordance with teachings of this disclosure. The blowdown system 100 of the illustrated example includes a relief line (e.g., a bypass line, a relief pipe, etc.) 102 that is fluidly coupled to a fluid distributor 104, which is a pipeline in this example, a deodorizer 106 and an outlet (e.g., an outlet section, an outlet portion, an outlet region, an outlet assembly, etc.) 108. In turn, the example relief line 102 includes a valve 110, a bleed line 112 and an orifice plate (e.g., an adjustable orifice plate) 114. The deodorizer 106 includes a block or housing 116 and is fluidly coupled to the aforementioned first bleed line 112 and a deodorant line 118 that extends to a reaction chamber 120, in this example. The example deodorant line 118 extends to a distribution pipe 122 and a sprayer 124, both of which are proximally located or positioned on the outlet 108 and/or the reaction chamber 120. In some examples, a blowdown controller 130 is implemented.

In operation, the example relief line 102 extends from the fluid distributor 104, which is implemented to transport a fluid (e.g., natural gas). In the illustrated example, the valve 110 is operated (e.g., manually, by the blowdown controller) to divert at least some of the fluid away from the fluid distributor 104. In turn, the fluid is directed to pass through the relief line 102, through the orifice plate 114 and into the reaction chamber 120. Further, the fluid is moved toward the deodorizer 106 via the first outlet line 112, thereby causing deodorant to exit the deodorizer 106 via the deodorant line 118 and, in turn, through the distribution pipe 122 and out the sprayer 124. As a result, the deodorant is sprayed onto the fluid flowing through and out of the reaction chamber 120.

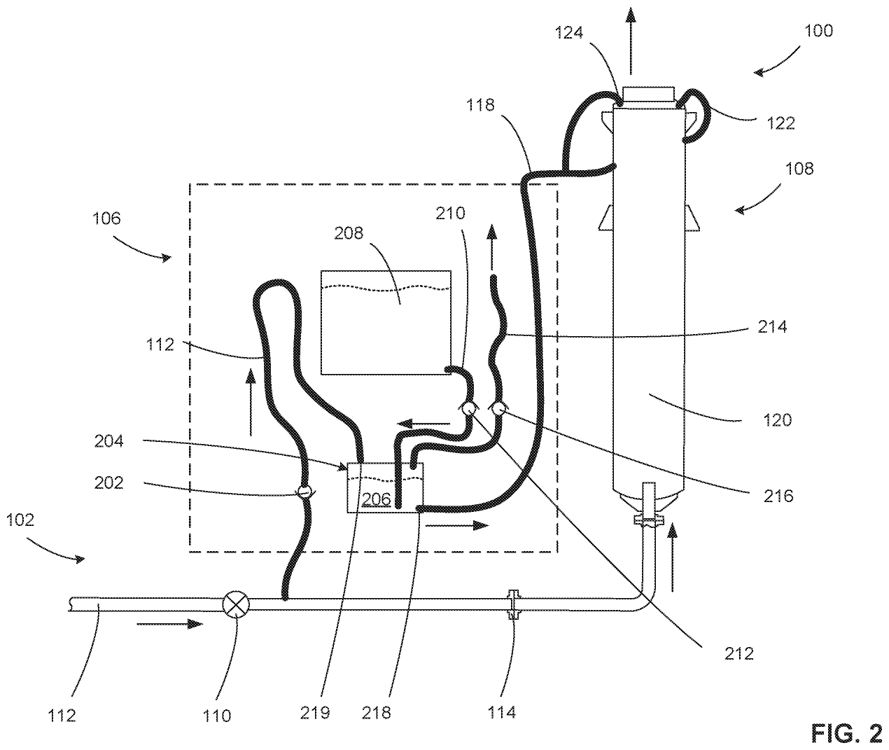

FIG. 2 is a detailed view of the example blowdown system 100 of FIG. 1. In the illustrated example of FIG. 2, the relief line 102 is shown operatively coupled to the valve 110 and the reaction chamber 120. Further, the bleed line 112 extends from and/or branches way from the relief line 102 and into the deodorizer 106. The example bleed line 112 includes a check valve 202 and is fluidly coupled to a pressure tank (e.g., a pressurized fluid tank, a pressure container, etc.) 204, which includes and/or stores a liquid deodorant 206. In this example, a storage tank (e.g., a bulk storage tank) 208 is fluidly coupled to the pressure tank 204 via a transfer line 210 with a respective a check valve 212. Further, a vent line (e.g., a pressure vent line) 214 and a check valve 216 are fluidly coupled to the pressure tank 204.

In the illustrated example, an exit port 218 of the pressure tank 204 is associated with the deodorant line 118 is positioned at a bottom portion (e.g., a bottom end) of the pressure tank 204. Further, an entry port (e.g., an inlet) 219 associated with the bleed line 112 is positioned at an upper portion (e.g., a top end) of the of the pressure tank 204. Accordingly, the pressure tank 204 is fluidly coupled to the deodorant line 112 and, thus, the distribution pipe 122 as well as the sprayer 124 mounted to the reaction chamber 120.

To deliver fluid from the fluid distributor 104 to the outlet 108 during a blowdown event, the valve 110 is opened, and a pressure of the fluid causes the fluid to flow toward the reaction chamber 120 and, in turn, exit from the outlet 108. In the illustrated example, the fluid is urged toward the orifice plate 114, which includes an opening or aperture that is used to generate a choked flow condition of the fluid moving through the fluid distributor 104 (shown in FIG. 1). In particular, the static pressure of the fluid is significantly greater upstream of the orifice plate 114 in comparison to downstream of the orifice plate 114.

To provide the deodorant 206 to the fluid from the pipeline and, thus, deodorize the fluid, the fluid from the fluid distributor 104 is directed to the outlet line 112 when the valve 110 is opened. In particular, the fluid is brought to a top portion or area of the pressure tank 204 via the entry port 219. As a result, the fluid urges the deodorant 206 into the deodorant line 118 that is fluidly coupled to a lower portion or area of the pressure tank 204 at the exit port 218 and, in turn, the distribution pipe 122 and the sprayer 124. In other words, the fluid from the fluid distributor 104 is provided to the pressure tank at a relatively higher position (in the view of FIG. 2) than the exit port 218 of the pressure tank 204. In this example, the deodorant 206 is sprayed onto the fluid from the relief line 102 as the fluid exits the reaction chamber 120. In particular, the deodorant 206 is sprayed onto the fluid as a fine mist so that the deodorant 206 evaporates onto the fluid in relatively quick manner and, as a result, marcaptans of the fluid are deodorized. In this example, the check valve 212 is closed during the aforementioned blowdown event. Further, during the blowdown event, the check valve 216 opens when a pressure of the pressure tank 204 is less than a threshold (e.g., less than 3 pounds per square inch (psi) greater than atmospheric pressure).

To refill the pressurized gas tank 204 with the deodorant 206, the storage tank 208 provides the deodorant 206 to the transfer line 210. Particularly, the check valve 212 of the transfer line 210 only enables flow of the deodorant 206 from the storage tank 208 to the pressure tank 204. In some examples, the storage tank 208 is positioned above the pressure tank 204 to facilitate filling thereof with minimal and/or no equipment (e.g., without pumps). In some such examples, gravity and/or a static pressure is used to fill the pressure tank 204.

During a non-blowdown event, the check valve 212 opens to fill pressure tank 204 with the deodorant 206 and excess fluid and/or gas flows through the aforementioned vent line 214 when the check valve 216 opens. In particular, an opening of the vent line 214 and/or the vent line 214 is positioned above (in the view of FIG. 2) the pressure tank 204. Further, the check valve 202 closes, thereby preventing the fluid from entering the pressure tank 204. In this example, the bleed line 112 enters the pressure tank 204 at a height and/or level above a maximum height of a fill line of the deodorant 206. Accordingly, the example blowdown system 100 is stagnant until the next blowdown event occurs.

In some examples, the deodorant 206 includes a liquid deodorant, such as Bioworld Odor Neutralizer (BON). However, any other appropriate type of deodorant can be implemented instead. In some examples, the liquid deodorant is diluted with water based on a dilution ratio of 3.1% to 10% of liquid deodorant (e.g., BON) to water. Additionally or alternatively, the deodorant is dosed based on Equation 1 below:

$$Amount of Liquid Deoderant [ GPM ] = .00441 * {P}^{0.4697} * \frac{B}{128} \qquad ( 1 )$$

where GPM is equal to gallons per minute, where P is equal to a pressure of the pressure tank 204 (e.g., in PSIG), and where B is equal to an amount of liquid deodorant (BON) added for a total of gallon of liquid deodorant solution. In some examples where the fluid is natural gas, air is added to the natural gas via a fan (e.g., via fan that provides air at least 4000 cubic feet per minute (cfm)). In some examples, an access point of the fluid distributor 104 is integrated with the orifice plate 114. In other examples, the access point is located in the reaction chamber 120 (e.g., an exit of the reaction chamber 120) and/or the relief line 102.

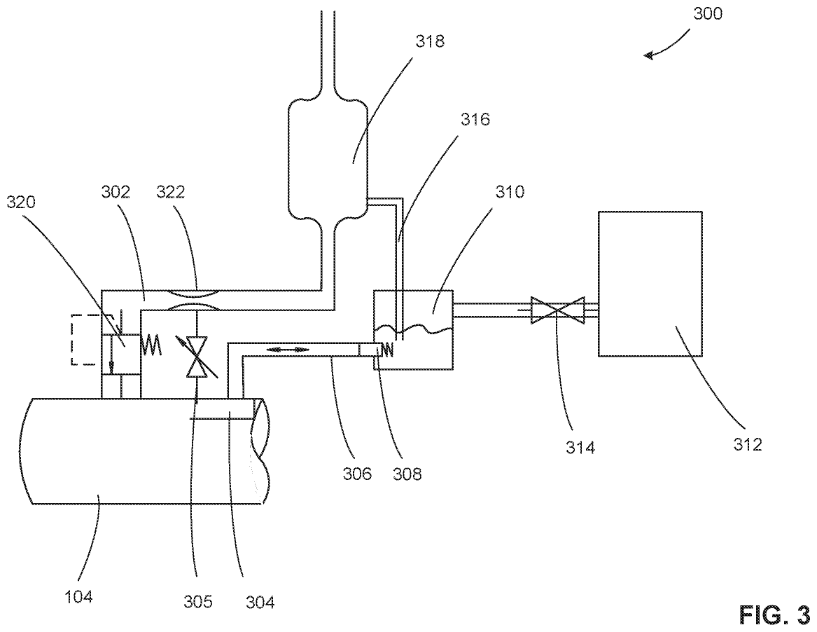

FIG. 3 illustrates an alternative example blowdown system 300. The blowdown system 300 of the illustrated example includes a relief line 302, a fluid tap 304, a gate valve 305, a bleed line 306, a pressure piston 308, a pressure tank 310, a storage tank 312, a refill valve (e.g., a refill check valve) 314, a deodorant line 316, and a reaction chamber 318. In some examples, the blowdown system 300 includes a pressure relief valve 320 and a nozzle (e.g., a venturi nozzle) 322.

In operation, during a blowdown event, the relief valve 320 is opened and, as a result, fluid from the fluid distributor 104 enters the relief line 302 and passes through the nozzle 322 before passing through the reaction chamber 318 and exiting the blowdown system 300. Further, the gate valve 305 is opened, thereby causing the fluid to move through the bleed line 306 and push the pressure piston 308. In turn, the pressure piston 308 is moved, thereby causing the fluid to urge deodorant stored in the pressure tank 310 to the deodorant line 316 and, thus, to the reaction chamber 318. As a result, the fluid from the relief line 302 that emerges from the nozzle 322 is mixed with the deodorant in the reaction chamber 318 prior to exiting the blowdown system 300. In this example, the storage tank 312 provides the deodorant to the pressure tank 310 when the refill valve 314 is opened. In this example, the refill valve 314 is closed when the deodorant is being provided to the reaction chamber 318.

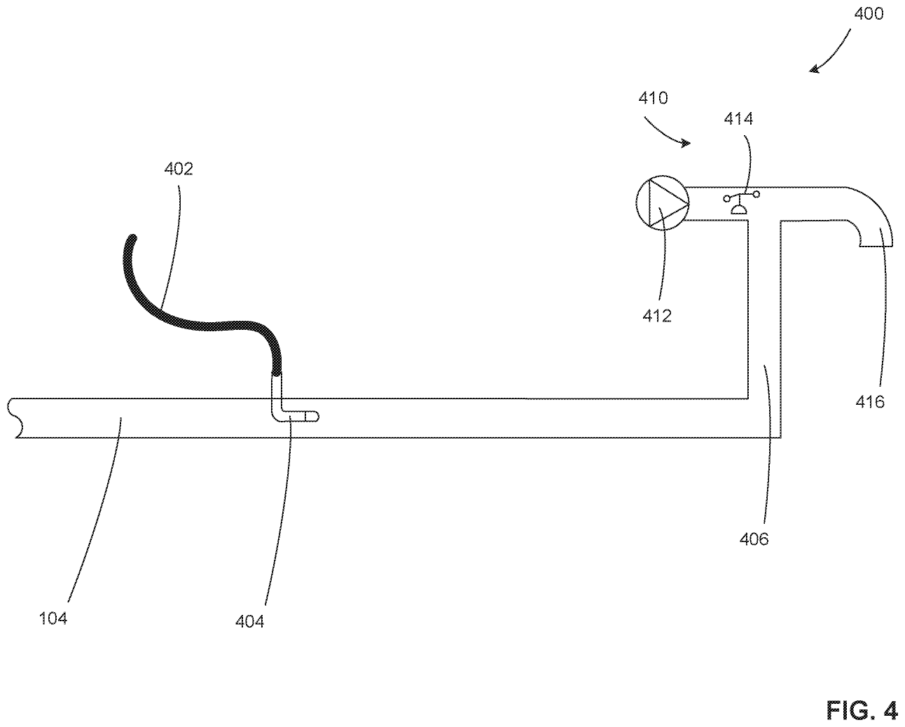

FIG. 4 illustrates another alternative example blowdown system 400. In contrast to the example blowdown systems 100 and 300 of FIGS. 1-3, the blowdown system 400 implements direct injection of the fluid distributor 104 with deodorant, instead. In the illustrated example, a fluid deodorant line 402 extends into an interior of the fluid distributor 104. Particularly, a nozzle 404 extends from a distal end of the deodorant line 402 to spray and/or deliver the deodorant to the fluid, which is natural gas in this example.

In this example, the blowdown system 400 includes a relief line 406 extending into a blowout apparatus 410. The example blowout apparatus includes a fan (e.g., an air mixing fan, a blower, etc.) 412, a pressure switch 414 and an outlet 416. The blowout apparatus 410 may be implemented to discharge deodorized fluid from the fluid distributor 104.

In operation, the deodorant is injected into the fluid distributor 104 via the nozzle 404. Further, the pressure switch 414 is opened and the fan 412 is operated to mix air into the fluid. As a result, a deodorized fluid air mixture exits from the outlet 416.

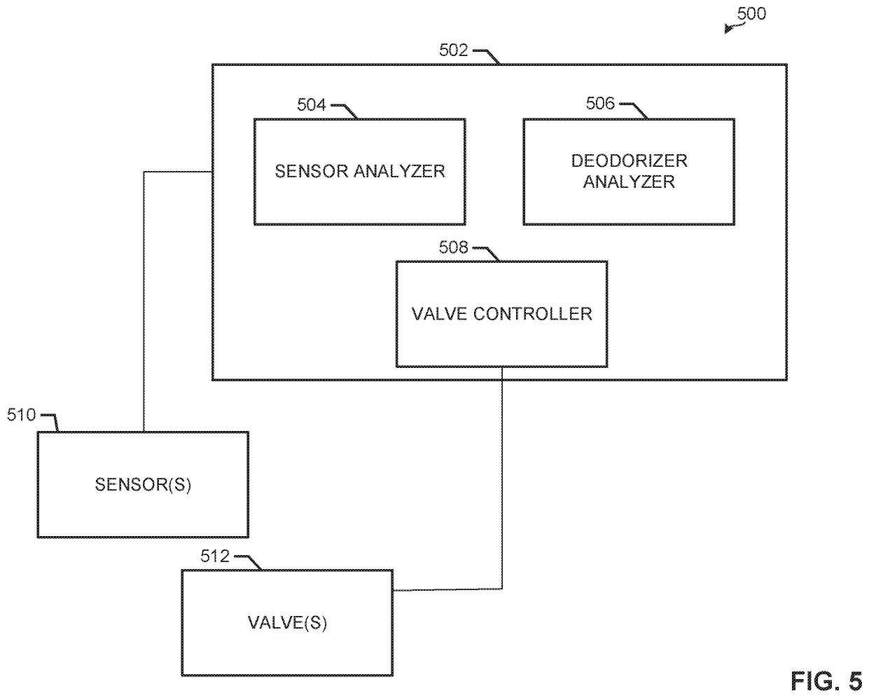

FIG. 5 is a schematic overview of an example blowdown control system 500 that can be implemented in examples disclosed herein. The example blowdown control system 500 may be be implemented in the blowdown controller 130 shown in FIG. 1 and includes a deodorant application analyzer 502 which, in turn includes a sensor analyzer 504, a deodorizer analyzer 506, and a valve controller 508. In this example, the deodorant application analyzer 502 is communicatively coupled to a sensor(s) 510 and a valve(s) 512, which can include any of the valves described above in connection with FIGS. 1-4.

The sensor analyzer 504 utilizes sensor data from the sensor(s) 510. For example, the sensor analyzer 504 can utilize pressure sensor data of the fluid distributor 104, fluid levels (e.g., stored fluid levels, etc.) and/or positions of the valve(s) 512 to determine a condition corresponding to whether a blowdown event should be initiated and/or a time to initiate the blowdown event. In other examples, the sensor(s) 510 measure marcaptans of fluid of the fluid distributor 104 (e.g., as colorimetric gas detector tubes) for a control loop related to reducing potentially detected odors of the fluid.

The deodorizer analyzer 506 can be implemented to determine whether deodorant should be applied to fluid from the fluid distributor 104. Additionally or alternatively, the deodorizer analyzer 506 can determine an amount of deodorant to be applied to fluid flowing out from the fluid distributor 104 during the blowdown event.

In some examples, the valve controller 508 is implemented to control the valve(s) 512. Particularly, the valve(s) 512 can be opened or closed by the valve controller 508 in a controlled sequence to initiate a blowdown event. Additionally or alternatively, the valve controller 508 coordinates movements of the valve(s) 512 during the blowdown event and/or in preparation for the blowdown event (e.g., tank filling, relief venting, pressurization, etc.).

While an example manner of implementing the blowdown control system 500 of FIG. 5 is illustrated in FIG. 5, one or more of the elements, processes and/or devices illustrated in FIG. 5 may be combined, divided, re-arranged, omitted, eliminated and/or implemented in any other way. Further, the example sensor analyzer 504, the example deodorizer analyzer 506, the example valve controller 508 and/or, more generally, the example blowdown control system 500 of FIG. 5 may be implemented by hardware, software, firmware and/or any combination of hardware, software and/or firmware. Thus, for example, any of the example sensor analyzer 504, the example deodorizer analyzer 506, the example valve controller 508 and/or, more generally, the example blowdown control system 500 could be implemented by one or more analog or digital circuit(s), logic circuits, programmable processor(s), programmable controller(s), graphics processing unit(s) (GPU(s)), digital signal processor(s) (DSP(s)), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example, sensor analyzer 504, the example deodorizer analyzer 506, and/or the example the example valve controller 508 is/are hereby expressly defined to include a non-transitory computer readable storage device or storage disk such as a memory, a digital versatile disk (DVD), a compact disk (CD), a Blu-ray disk, etc. including the software and/or firmware. Further still, the example blowdown control system 500 of FIG. 5 may include one or more elements, processes and/or devices in addition to, or instead of, those illustrated in FIG. 5, and/or may include more than one of any or all of the illustrated elements, processes and devices. As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

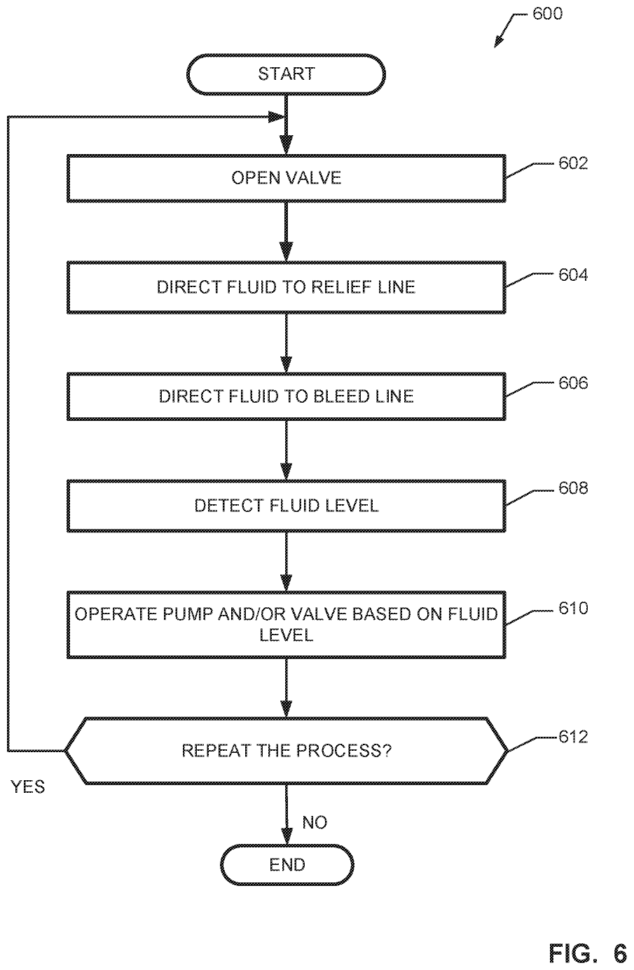

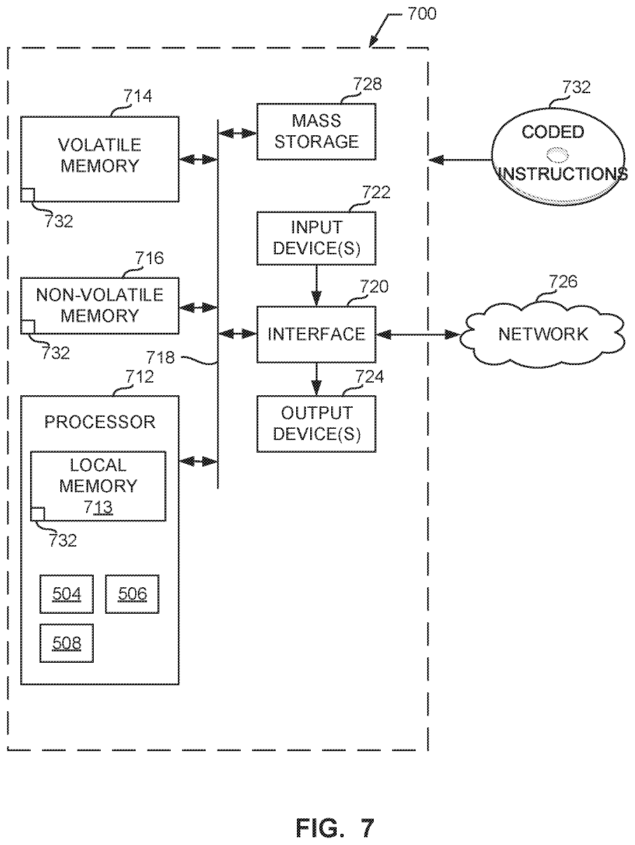

A flowchart representative of example hardware logic, machine readable instructions, hardware implemented state machines, and/or any combination thereof for implementing the blowdown control system 500 of FIG. 5 is shown in FIG. 6. The machine readable instructions may be one or more executable programs or portion(s) of an executable program for execution by a computer processor such as the processor 712 shown in the example processor platform 700 discussed below in connection with FIG. 7.

The program may be embodied in software stored on a non-transitory computer readable storage medium such as a CD-ROM, a floppy disk, a hard drive, a DVD, a Blu-ray disk, or a memory associated with the processor 712, but the entire program and/or parts thereof could alternatively be executed by a device other than the processor 712 and/or embodied in firmware or dedicated hardware. Further, although the example program is described with reference to the flowchart illustrated in FIG. 6, many other methods of implementing the example blowdown control system 500 may alternatively be used. For example, the order of execution of the blocks may be changed, and/or some of the blocks described may be changed, eliminated, or combined. Additionally or alternatively, any or all of the blocks may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine readable instructions described herein may be stored in one or more of a compressed format, an encrypted format, a fragmented format, a compiled format, an executable format, a packaged format, etc. Machine readable instructions as described herein may be stored as data (e.g., portions of instructions, code, representations of code, etc.) that may be utilized to create, manufacture, and/or produce machine executable instructions. For example, the machine readable instructions may be fragmented and stored on one or more storage devices and/or computing devices (e.g., servers). The machine readable instructions may require one or more of installation, modification, adaptation, updating, combining, supplementing, configuring, decryption, decompression, unpacking, distribution, reassignment, compilation, etc. in order to make them directly readable, interpretable, and/or executable by a computing device and/or other machine. For example, the machine readable instructions may be stored in multiple parts, which are individually compressed, encrypted, and stored on separate computing devices, wherein the parts when decrypted, decompressed, and combined form a set of executable instructions that implement a program such as that described herein.

In another example, the machine readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine readable instructions may need to be configured (e.g., settings stored, data input, network addresses recorded, etc.) before the machine readable instructions and/or the corresponding program(s) can be executed in whole or in part. Thus, the disclosed machine readable instructions and/or corresponding program(s) are intended to encompass such machine readable instructions and/or program(s) regardless of the particular format or state of the machine readable instructions and/or program(s) when stored or otherwise at rest or in transit.

The machine readable instructions described herein can be represented by any past, present, or future instruction language, scripting language, programming language, etc. For example, the machine readable instructions may be represented using any of the following languages: C, C++, Java, C #, Perl, Python, JavaScript, HyperText Markup Language (HTML), Structured Query Language (SQL), Swift, etc.

As mentioned above, the example processes of FIG. 7 may be implemented using executable instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc. may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, and (7) A with B and with C. As used herein in the context of describing structures, components, items, objects and/or things, the phrase “at least one of A and B” is intended to refer to implementations including any of (1) at least one A, (2) at least one B, and (3) at least one A and at least one B. Similarly, as used herein in the context of describing structures, components, items, objects and/or things, the phrase “at least one of A or B” is intended to refer to implementations including any of (1) at least one A, (2) at least one B, and (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, and (3) at least one A and at least one B. Similarly, as used herein in the context of describing the performance or execution of processes, instructions, actions, activities and/or steps, the phrase “at least one of A or B” is intended to refer to implementations including any of (1) at least one A, (2) at least one B, and (3) at least one A and at least one B.

As used herein, singular references (e.g., “a”, “an”, “first”, “second”, etc.) do not exclude a plurality. The term “a” or “an” entity, as used herein, refers to one or more of that entity. The terms “a” (or “an”), “one or more”, and “at least one” can be used interchangeably herein. Furthermore, although individually listed, a plurality of means, elements or method actions may be implemented by, e.g., a single unit or processor. Additionally, although individual features may be included in different examples or claims, these may possibly be combined, and the inclusion in different examples or claims does not imply that a combination of features is not feasible and/or advantageous.

The example method 600 of FIG. 6 begins as a blowdown event of a pipeline system is to be initiated. In this example, the blowdown event corresponds to the release of natural gas from a pipeline (e.g., the fluid distributor 104) into the atmosphere. The natural gas is to be deodorized.

At block 602, a valve (e.g., the valve 110) is opened to initiate the blowdown event. In this example, the valve is opened manually (e.g., by an operator). However, in some other examples, the valve controller 508 directs movement of and/or controls the valve.

At block 604, the fluid from the pipeline is directed to a relief line (e.g., the bleed line 102). In this example, the fluid is directed to a reaction chamber before exiting the reaction chamber and entering an external environment.

At block 606, the fluid from the pipeline is directed to a relief line (e.g., the relief line 102). In turn, the fluid directed to the relief line pressurizes deodorant in a pressure tank (e.g., the pressure tank 208) so that the deodorant is urged toward an outlet of the reaction chamber.

At block 608, in some examples, a sensor (e.g., the sensor 510) is implemented to detect a fluid level of deodorant in the aforementioned pressure tank. For example, the sensor analyzer 504 analyzes an amount of deodorant to be stored in the pressure tank to adequately deodorize the fluid leaving the reaction chamber. Additionally or alternatively, the deodorant analyzer 506 determines an amount of the deodorant to be applied to the fluid based on sensor data.

At block 610, a pump or other device (e.g., a valve) is operated by the valve controller 508, for example, to control the fluid level of the pressure tank.

At block 612, it is determined whether to repeat the process. If the process is to be repeated (block 612), control of the process returns to block 602. Otherwise, the process ends.

FIG. 7 is a block diagram of an example processor platform 700 structured to execute the instructions of FIG. 6 to implement the blowdown control system 500 of FIG. 5. The processor platform 700 can be, for example, a server, a personal computer, a workstation, a self-learning machine (e.g., a neural network), a mobile device (e.g., a cell phone, a smart phone, a tablet such as an iPad′), a personal digital assistant (PDA), an Internet appliance, a DVD player, a CD player, a digital video recorder, a Blu-ray player, a gaming console, a personal video recorder, a set top box, a headset or other wearable device, or any other type of computing device.

The processor platform 700 of the illustrated example includes a processor 712. The processor 712 of the illustrated example is hardware. For example, the processor 712 can be implemented by one or more integrated circuits, logic circuits, microprocessors, GPUs, DSPs, or controllers from any desired family or manufacturer. The hardware processor may be a semiconductor based (e.g., silicon based) device. In this example, the processor implements the example sensor analyzer 504, the example deodorizer analyzer 506 and the example valve controller 508.

The processor 712 of the illustrated example includes a local memory 713 (e.g., a cache). The processor 712 of the illustrated example is in communication with a main memory including a volatile memory 714 and a non-volatile memory 716 via a bus 718. The volatile memory 714 may be implemented by Synchronous Dynamic Random Access Memory (SDRAM), Dynamic Random Access Memory (DRAM), RAMBUS® Dynamic Random Access Memory (RDRAM®) and/or any other type of random access memory device. The non-volatile memory 716 may be implemented by flash memory and/or any other desired type of memory device. Access to the main memory 714, 716 is controlled by a memory controller.

The processor platform 700 of the illustrated example also includes an interface circuit 720. The interface circuit 720 may be implemented by any type of interface standard, such as an Ethernet interface, a universal serial bus (USB), a Bluetooth® interface, a near field communication (NFC) interface, and/or a PCI express interface.

In the illustrated example, one or more input devices 722 are connected to the interface circuit 720. The input device(s) 722 permit(s) a user to enter data and/or commands into the processor 712. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 724 are also connected to the interface circuit 720 of the illustrated example. The output devices 724 can be implemented, for example, by display devices (e.g., a light emitting diode (LED), an organic light emitting diode (OLED), a liquid crystal display (LCD), a cathode ray tube display (CRT), an in-place switching (IPS) display, a touchscreen, etc.), a tactile output device, a printer and/or speaker. The interface circuit 720 of the illustrated example, thus, typically includes a graphics driver card, a graphics driver chip and/or a graphics driver processor.

The interface circuit 720 of the illustrated example also includes a communication device such as a transmitter, a receiver, a transceiver, a modem, a residential gateway, a wireless access point, and/or a network interface to facilitate exchange of data with external machines (e.g., computing devices of any kind) via a network 726. The communication can be via, for example, an Ethernet connection, a digital subscriber line (DSL) connection, a telephone line connection, a coaxial cable system, a satellite system, a line-of-site wireless system, a cellular telephone system, etc.

The processor platform 700 of the illustrated example also includes one or more mass storage devices 728 for storing software and/or data. Examples of such mass storage devices 728 include floppy disk drives, hard drive disks, compact disk drives, Blu-ray disk drives, redundant array of independent disks (RAID) systems, and digital versatile disk (DVD) drives.

The machine executable instructions 732 of FIG. 6 may be stored in the mass storage device 728, in the volatile memory, in the non-volatile memory 716, and/or on a removable non-transitory computer readable storage medium such as a CD or DVD.

Example 1 includes an apparatus having a valve to enable the fluid to enter a relief line from the fluid distributor, the relief line including an outlet, a bleed line to receive the fluid, the bleed line fluidly coupled to the relief line, a pressure tank fluidly coupled to the bleed line, the pressure tank to store a deodorant, and a deodorant line fluidly coupled between the pressure tank and the outlet, the fluid in the bleed line to urge the deodorant to the deodorant line.

Example 2 includes the apparatus as defined in example 1, where the outlet includes a reaction chamber.

Example 3 includes the apparatus as defined in example 2, further including a sprayer or injector, the sprayer or injector fluidly coupled to the deodorant line and preferably arranged to spray/inject into the reaction chamber and most preferably arranged proximate the outlet.

Example 4 includes the apparatus as defined in any of examples 1 or 2, further including a sprayer or injector positioned proximate the outlet, the sprayer or injector fluidly coupled to the deodorant line.

Example 5 includes the apparatus as defined in any of examples 1 to 4, further including a storage tank fluidly coupled to the pressure tank, the storage tank to store the deodorant and being in an elevated position above the pressure tank.

Example 6 includes the apparatus as defined in any of examples 1 to 5, further including a relief valve operatively coupled to the pressure tank.

Example 7 includes the apparatus as defined in any of examples 1 to 6, where the valve includes a gate valve.

Example 8 includes the apparatus as defined in any of examples 1 to 7, further including a venturi nozzle operatively coupled between the relief line and a reaction chamber of the outlet.

Example 9 includes the apparatus as defined in any of examples 1 to 8, further including a pressure piston operatively coupled between the bleed line and the pressure tank.

Example 10 includes a method. The method includes opening a valve of a relief line that is fluidly coupled to a fluid distributor, directing fluid from the relief line to an outlet, and directing fluid from the relief line to a bleed line and into a pressure tank that stores a deodorant, the fluid from the bleed line to urge the deodorant toward the fluid of the relief line.

Example 11 includes the method as defined in example 10, further including providing the fluid to the pressure tank via a storage tank elevated above the pressure tank.

Example 12 includes the method as defined in any of examples 10 or 11, where the deodorant is to move to the outlet via a sprayer or injector.

Example 13 includes the method as defined in any of examples 10 to 12, where the directing the fluid to the relief line includes directing the fluid to a reaction chamber.

Example 14 includes the method as defined in claim 13, where the deodorant is directed to the reaction chamber via a sprayer or injector prior to exiting the outlet.

Example 15 includes the method as defined in any of examples 10 to 14, further including detecting, via a sensor, a fluid level of the pressure tank, and operating, based on instructions executed by a processor, a pump that supplies deodorant to the pressure tank based on detected fluid level.

Example 16 includes a blowdown system to be used with a fluid distributor. The blowdown system includes a relief line, a valve to enable fluid to flow from the fluid distributor and into a relief line, a reaction chamber fluidly coupled to the relief line, and a pressure tank fluidly coupled to the bleed line, the pressure tank to store a deodorant to be supplied to a nozzle of the reaction chamber, the deodorant to be urged toward the nozzle by the fluid in the bleed line.

Example 17 includes the blowdown system as defined in example 16, further including a storage tank to provide the deodorant to the pressure tank.

Example 18 includes the blowdown system as defined in any of examples 16 or 17, further including an adjustable orifice plate of the relief line.

Example 19 includes the blowdown system as defined in any of examples 16 to 18, where an inlet of the pressure tank corresponding to the bleed line is located at an upper portion of the pressure tank, and where an outlet of the pressure tank corresponding to a deodorant line is located a lower portion of the pressure tank.

Example 20 includes the blowdown system as defined in any of examples 16 to 19, further including a venturi nozzle operatively coupled between the relief line and the reaction chamber.

Example 21 includes the blowdown system as defined in any of examples 16 to 20, where the reaction chamber includes a silencer.

Example 22 includes the blowdown system as defined in any of examples 16 to 21, wherein the reaction chamber is positioned within a silencer.

Example 23 includes the blowdown system as defined in any of examples 16 to 22, where the fluid includes natural gas.

From the foregoing, it will be appreciated that example methods, apparatus and articles of manufacture have been disclosed that enable effective deodorization of fluids, such as a gas. Examples disclosed herein can also be implemented in a cost-effective manner by reducing and/or eliminating typical fluid devices (e.g., pumps, etc.).

This patent claims priority to, and the benefit of, U.S. Provisional Patent Application Ser. No. 62/854,679 and U.S. Provisional Patent Application Ser. No. 62/854,680, both of which were filed on May 30, 2019. U.S. Provisional Patent Application Ser. No. 62/854,679 and U.S. Provisional Patent Application Ser. No. 62/854,680 are hereby incorporated herein by reference in its entirety.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent. While examples disclosed herein are shown in the context of fluid pipelines, examples disclosed herein can be applied to any appropriate example corresponding to adding a fluid to another fluid.

The following claims are hereby incorporated into this Detailed Description by this reference, with each claim standing on its own as a separate embodiment of the present disclosure.

What is claimed is:

1. An apparatus for deodorizing fluid from a fluid distributor, the apparatus comprising:
a valve to enable the fluid to enter a relief line from the fluid distributor, the relief line including an outlet;
a bleed line to receive the fluid, the bleed line fluidly coupled to the relief line;
a pressure tank fluidly coupled to the bleed line, the pressure tank to store a deodorant; and
a deodorant line fluidly coupled between the pressure tank and the outlet, the fluid in the bleed line to urge the deodorant to the deodorant line.

2. The apparatus as defined in claim 1, wherein the outlet includes a reaction chamber.

3. The apparatus as defined in claim 2, further including a sprayer or injector, the sprayer or injector fluidly coupled to the deodorant line and preferably arranged to spray/inject into the reaction chamber and most preferably arranged proximate the outlet.

4. The apparatus as defined in claim 1, further including a sprayer or injector positioned proximate the outlet, the sprayer or injector fluidly coupled to the deodorant line.

5. The apparatus as defined in claim 1, further including a storage tank fluidly coupled to the pressure tank, the storage tank to store the deodorant and being in an elevated position above the pressure tank.

6. The apparatus as defined in claim 1, further including a relief valve operatively coupled to the pressure tank.

7. The apparatus as defined in claim 1, wherein the valve includes a gate valve.

8. The apparatus as defined in claim 1, further including a venturi nozzle operatively coupled between the relief line and a reaction chamber of the outlet.

9. The apparatus as defined in claim 1, further including a pressure piston operatively coupled between the bleed line and the pressure tank.

10. A method comprising
opening a valve of a relief line that is fluidly coupled to a fluid distributor,
directing fluid from the relief line to an outlet; and
directing fluid from the relief line to a bleed line and into a pressure tank that stores a deodorant, the fluid from the bleed line to urge the deodorant to flow toward the fluid of the relief line.

11. The method as defined in claim 10, further including providing the fluid to the pressure tank via a storage tank elevated above the pressure tank.

12. The method as defined in claim 10, wherein the deodorant is to move to the outlet via a sprayer or injector.

13. The method as defined in claim 10, wherein the directing the fluid to the relief line includes directing the fluid to a reaction chamber.

14. The method as defined in claim 13, wherein the deodorant is directed to the reaction chamber via a sprayer or injector prior to exiting the outlet.

15. The method as defined in claim 10, further including:
detecting, via a sensor, a fluid level of the pressure tank; and
operating, based on instructions executed by a processor, a pump that supplies deodorant to the pressure tank based on detected fluid level.

16. A blowdown system to be used with a fluid distributor, the blowdown system comprising:
a relief line;
a valve to enable fluid to flow from the fluid distributor and into a relief line;
a bleed line fluidly coupled to the relief line;
a reaction chamber fluidly coupled to the relief line; and
a pressure tank fluidly coupled to the bleed line, the pressure tank to store a deodorant to be supplied to a nozzle of the reaction chamber, the deodorant to be urged toward the nozzle by the fluid in the bleed line.

17. The blowdown system as defined in claim 16, further including a storage tank to provide the deodorant to the pressure tank.

18. The blowdown system as defined in claim 16, further including an adjustable orifice plate of the relief line.

19. The blowdown system as defined in claim 16, wherein an inlet of the pressure tank corresponding to the bleed line is located at an upper portion of the pressure tank, and wherein an outlet of the pressure tank corresponding to a deodorant line is located a lower portion of the pressure tank.

20. The blowdown system as defined in claim 16, further including a venturi nozzle operatively coupled between the relief line and the reaction chamber.

21. The blowdown system as defined in claim 16, wherein the reaction chamber includes a silencer

22. The blowdown system as defined in claim 16, wherein the reaction chamber is positioned within a silencer.

23. The blowdown system as defined in claim 16, wherein the fluid includes natural gas.
